(19) United States
(12) Patent Application Publication
(10) Pub. No.: US 2014/0316010 A1
(43) Pub. Date: Oct. 23, 2014

(54) COMPOSITION FOR THE TREATMENT OF DANDRUFF

(71) Applicant: Chi Wu Hsu, New Taipei City (TW)

(72) Inventor: Chi Wu Hsu, New Taipei City (TW)

(21) Appl. No.: 14/252,826

(22) Filed: Apr. 15, 2014

(30) Foreign Application Priority Data
Apr. 19, 2013 (TW) 102114056

(51) Int. Cl. A61K 8/86; A61Q 5/00; A61K 8/60; A61K 8/34

(52) U.S. Cl. CPC A61K 8/86; A61K 8/345; A61Q 5/006; A61K 8/60
USPC 514/723; 514/738

(57) ABSTRACT

A composition for treating dandruff is provided. The composition includes glycerol with a concentration of not larger than 10 wt %, a second, low molecular weight polyol with a concentration of not larger than 10 wt %, and a saline solution. The composition is free of chemical surfactants, antiseptics, or foaming agents and the like, which may cause allergic or hypersensitive conditions, and has the advantages of simple constituents, low cost, and effective outcomes on the treatment of dandruff. Long-term application of the composition on the scalp is therefore suggested.

### CROSS REFERENCE TO RELATED APPLICATIONS

This non-provisional application claims priority claim under 35 U.S.C. §119(a) on Patent Application No. 102114056 filed in Taiwan, R.O.C. on Apr. 19, 2013, the entire contents of which are hereby incorporated by reference herein.

### BACKGROUND OF THE INVENTION

1. Field of the Invention

The present invention relates to a formula for treating skin desquamation and, more particularly, to a composition for effectively treating dandruff on the scalp.

2. Description of Related Art

Dermatitis, or eczema, refers to any inflammatory skin conditions, and its symptoms vary with different forms ranging from pustules to rashes to exudates to exfoliated skin eruptions to eschar skin disorders, all are likely to be associated with severe itching, stinging sensation in the skin. It may cause a negative effect to the patients of dermatitis on the quality of life and the health of mind and body, and therefore the impact cannot be underestimated.

Stratum corneum (SC) is the outermost layer of the epidermis, and its function is to provide a barrier to protect underlying tissues from infection and dehydration. It takes about one month for a normal human skin to grow and mature, after the period the skin goes through a metabolic process of ageing SC, during which the skin desquamates without being noticed by human eyes. In general, when the ambient relative humidity is lower than 50%, SC hydration will drop below 10%, causing the SC to lose flexibility and further to desquamate from the skin due to dryness, and therefore causes damage to, or inflammation on, the skin. Typical dermatitis can be relieved by topically applying the ointment containing steroids, such as Cort. S and Cutivate, on affected portions and, at the same time, avoiding as many stimuli in the environment as possible, for example, using non-irritating cleaning products or bathing with clean water whenever possible.

Dandruff is, despite no harm to human health, a common problem of scalp that is irritating, but the symptoms of scalp itching and increasing skin scales often cause embarrassments and obstacles in interpersonal relationships. Dandruff can be caused by: seborrheic dermatitis, psoriasis, contact dermatitis caused by chemical materials (e.g., dyeing agents, hair gels, or hair waxes), fungal infection (ringworm), or not being shampooed for a long period. In addition, the formation of dandruff can be adversely accelerated by stress, nervousness, nutrition disorder, not being enough sleep, or tiredness.

Not like normal skins, the affected scalp of a patient with dandruff problem may develop, typically within 2 to 7 days in average, white bulky skin scales that are noticeable by human eyes. Seborrheic dermatitis is the main reason that causes dandruff on the scalp and, as a result, dandruff is the most common symptom found in a patient of seborrheic dermatitis. It is known that the formation of dandruff is related to overly breeding of fungi on the skin (i.e., Malassezia including Malassezia furfur and Malassezia globosa). Research also shows that the amount of fungi of Malassezia on the scalp where dandruff is found may be up to 1.5-2 times as many as that on normal skins. The fungi substitute the triglyceride of the sebum for the oleic acid through a metabolic process. The oleic acid may, after permeating into the SC of the epidermis, induce some sensitive skins to inflame, and further interfere with the biologic constancy of the SC cells, which adversely facilitates epidermal desquamation, thereby producing dandruff

Some shampoos with tar constituent have been proved to be effective to suppress dandruff caused by the diseases of scalp psoriasis and seborrheic dermatitis, but most of the shampoos that claim anti-dandruff are not as effective as claimed. The anti-dandruff shampoos on the market are primarily made up of: ketoconazole, zinc pyrithione (ZP), salicylic acid, selenium sulfide (SeS₂), ciclopirox olamine (CPO), piroctone olamine, and pyridinethione salts. More details regarding the constitutes can be referred to U.S. Pat. Nos. 4,835,148 and 6,538,011.

Studies indicate that, in any part of the human body, there exists fat among SC cells, where the fat forms a “bricks and mortar” morphology. The fatty molecules perpendicular to the skin surface are arranged in parallel (i.e., parallel fatty membrane), and the gap of wide-narrow-wide (by 4 nm-2 nm-4 nm) formed by each row of fatty molecules is shown to be the most effective morphology to prevent water inside the body from permeating outward (Bouwstra J A et al., J Lipid Res:39:186-196). If the morphology is damaged or changed, the function of preventing water from vaporizing will be diminished. Some chemical materials have been shown to destroy the normal packing pattern of the fat layer of the gap between the SC cells. These chemical materials, like low quality of emulsifiers and surfactants, dehydrate the skin or make the skin sensitive and, even worse, cause the skin to inflame, swell, or desquamate.

Another study (Hsu, “Effect of Sebum on Skin Barrier Function and Morphology,” M.S. Thesis, institute of Clinical Pharmacy, NCKU, Taiwan, 2009) shows related observation as follows. The results indicated that daily treatment of human sebum on the skin surface of hairless mice caused inflammation on the skin surface, increased skin scales, and increased the transepidermal water loss (TEWL), within one week, by 3-4 times; on the other hand, epidermal hydration was decreased by two third the normal skin without treatment. The skin of mice gradually returned to its normal skin barrier function within five days after cessation of treatment of human sebum. In addition, daily application of unsaturated fatty acid on the skin affects the skin barrier function, induces epidermal proliferation, and alters the intercellular membrane morphology in the SC, which suggested that unsaturated fatty acid had a negative effect on the treatment of dermatitis.

The approaches of the treatment of dandruff in the prior art focus on how to rinse scalps or suppress Malassezia. However, these approaches only “treat the symptoms,” but not “treat the root of the problem.” What is needed is to provide a medicament or composition that helps the scalp to return to its normal skin barrier function and that is, gentle in nature, suitable to be applied to newborn infants.

### BRIEF SUMMARY OF THE INVENTION

In view of the foregoing, an object of the present invention is to provide a composition for treating dandruff to improve anti-dandruff effects in the prior art. The composition forms a hydrogen bond by combing with the water molecule of the human body, in order to increase the effect of hydrating moisture so as to revive the skin.

The composition of the present invention includes glycerol with a concentration of not larger than 10 wt %, a second, low molecular weight polyol with a concentration of not larger than 10 wt %, and a saline solution with a concentration not larger than 10 wt %. The second, low molecular weight is any one, or a combination of any two or more than two, selected from the group consisting of propylene glycol (PG), 1,3-butylene glycol (BG), sorbital, and polyethylene glycol (PEG).

In one embodiment, the concentration of the glycerol is smaller than 5 wt %, and the total concentration of the second, low molecular weight polyol is smaller than 5 wt %.

In another embodiment, the concentration of the glycerol is 2-3 wt %, and the total concentration of the second, low molecular weight polyol is 2-3 wt %.

In still another embodiment, the concentration of the glycerol is 2-3 wt %, and the total concentration of the second, low molecular weight polyol is 2-3 wt %, where the second, low molecular weight polyol includes sorbital.

In addition to the treatment of dandruff, the composition of the present invention is applicable to a wide range of applications, including pharmaceutics, haircare products, cosmetic products, skincare products, and perfumes.

The advantageous effects of the present invention over prior-art approaches is that the application of the composition provides a self-recovery environment for the skin such that the fatty molecules in the gap of the SC cells can maintain normal packing pattern. Hence, the scalp keeps a normal level of hydration to prevent from inducing dandruff. Moreover, the composition is free of chemical surfactants, antiseptics, or foaming agents and the like, which may cause allergic or hypersensitive conditions, and has the advantages of simple constituents, low cost, effective outcomes on the treatment of dandruff.

### DETAILED DESCRIPTION OF THE PREFERRED EMBODIMENTS

The structure as well as a preferred mode of use, further objects, and advantages of the present invention will be best understood by referring to the following detailed description of some embodiments.

Instead of applying sebum on the skin surface as disclosed in the prior art, which could damage normal packing pattern of fatty molecules in the SC, and thus diminish the skin barrier function, the present invention provides a composition that is effective in the treatment of dandruff by maintaining a normal skin barrier function. The composition is made up of glycerol with not larger than 10 wt %, a (a second) low-molecular weight polyol with not larger than 10 wt %, and saline solution with not larger than 10 wt %.

Glycerol (scientific name as Propanetriol) is known as a propyl alcohol compound which is a soluble small molecule with moisterization. It is known that 1 gram of glycerol approximately absorbs 0.6 grams of water. The glycerol of the present invention can be selected from, but is not limited to, for example, synthetic glycerol, vegetable glycerol, fermented glycerol, or glycerol generated through a soap-making process. The concentration of the glycerol is not larger than 10 wt % of the composition, for example, the concentration can be 8 wt %, 1 wt %, or 0.1 wt %, preferably within 5 wt %, and more preferably within 2-3 wt % of the composition.

The second, low molecular weight polyol of the present invention can be, but is not limited to, any one, or a combination of at least two, selected from the group consisting of propylene glycol (PG), 1,3-butylene glycol (BG), sorbitol (molecular formula: C₆H₁₄O₆, including types of sorbitol and mannitol, which are enantiomers), and polyethylene glycol (PEG). Persons of ordinary skill in the art should be able to use any polyol of low-molecular weight and non-irritating to the skin. The total concentration of the second polyol is not larger than 10 wt % of the composition, for example, the concentration can be 10 wt %, 6 wt %, or 0.7 wt %, preferably within 5 wt %, and more preferably within 2-3 wt %.

In one embodiment of the present invention, the second, low molecular weight polyol is made up with PG and BG with appropriate proportions of total concentration of 2 wt % (e.g., 0.1 wt % of PG and 1.9 wt % of BG). In another embodiment, the second, low molecular weight polyol is made up with BG, sorbitol, and PEG with appropriate proportions of total concentration of 3 wt % (e.g., 1 wt % of BG, 1.6 wt % of sorbitol, and 0.4 wt % of PEG). In still another embodiment, the second, low molecular weight polyol is made up with PG, BG, sorbitol, and PEG with appropriate proportions of total concentration of 2.5 wt % (e.g., 0.2 wt % of PG, 1 wt % of BG, 1 wt % of sorbitol, and 0.3 wt % of PEG). In particularly another embodiment, the second, low molecular weight polyol only includes sorbitol with concentration of 2 wt %.

It should be noted that the embodiments are not intended to limit the scope of the present invention. The preferred embodiments described above serve to demonstrate the features of the present invention so that a person skilled in the art can understand and implement the present invention by adopting different concentrations, proportions, and combinations of the second, low molecular weight polyol accordingly.

The “low-molecular weight” in the present invention refers to a molecular weight of less than 400 Da. Considering the chemical property and applications, PEG, being a polymer molecule, is measured at a molecular weight of less than 600 Da.

Also, the ingredients of the composition of the present invention are pure in nature and harmless to the human body and skin. Persons of ordinary skills in the art can implement the composition according to the description and the embodiments disclosed.

The glycerol and the second, low molecular weight polyol are soluble in water, and water is the largest percentage of ingredient in the composition, and thus the composition of the present invention is a homogenous, transparent water solution by the appearance. Therefore, the application of the composition is harmless to the scalp and is suitable for long-term use, making the composition a safe and effective formula for anti-dandruff.

In addition to the treatment of dandruff, the composition of the present invention is applicable to a wide range of applications, including pharmaceutics, haircare products, cosmetic products, skincare products, and perfumes.

The composition (an aqueous solution) for the treatment of dandruff of the present invention is primarily based on the formation of hydrogen bonds so as to maintain normal packing pattern of fatty molecules in the gaps of the SC cells. Moreover, the composition has the advantages of simple constituents, low cost, and effective outcomes on the treatment of dandruff. Furthermore, the composition is free of surfactants, which are commonly used in typical haircare products, antiseptics, or foaming agents and the like, and therefore the composition is suitable for long-term use. It is sure that the scalp will not induce dandruff, or cause itching due to dryness, as long as the scalp hydration is kept at a normal level.

The embodiments described above serve to demonstrate the features of the present invention so that a person skilled in the art can understand the contents disclosed herein and implement the present invention accordingly. The embodiments, however, are not intended to limit the scope of the present invention, which is defined only by the appended claims. Therefore, all equivalent changes or modifications which do not depart from the spirit of the present invention should fall within the scope of the appended claims.

What is claimed is:

1. A composition for treating dandruff, comprising:
a glycerol with a concentration not larger than 10 wt %;
a second, low molecular weight polyol with a concentration not larger than 10 wt %; and
an aqueous solution;
wherein all weights being relative to the total weight of the composition.

2. The composition as of claim 1, wherein said second, low molecular weight polyol is any one, or a combination of at least two, selected from the group consisting of propylene glycol (PG), 1,3-butylene glycol (BG), sorbital, and polyethylene glycol (PEG).

3. The composition as of claim 1, wherein the total concentration of said second, low molecular weight polyol is 2-3 wt %.

4. The composition as of claim 2, wherein the total concentration of said second, low molecular weight polyol is 2-3 wt %.

5. The composition as of claim 1, wherein the concentration of said glycerol is 2-3 wt %, and the total concentration of said second, low molecular weight polyol is 2-3 wt %.

6. The composition as of claim 2, wherein the concentration of said glycerol is 2-3 wt %, and the total concentration of said second, low molecular weight polyol is 2-3 wt %.

7. The composition as of claim 1, wherein said second, low molecular weight polyol includes said sorbital.

8. The composition as of claim 2, wherein said second, low molecular weight polyol includes said sorbital.

9. The composition as of claim 3, wherein said second, low molecular weight polyol includes said sorbital.

10. The composition as of claim 4, wherein said second, low molecular weight polyol includes said sorbital.

11. The composition as of claim 9, wherein said second, low molecular weight polyol is said sorbital.

12. The composition as of claim 10, wherein said second, low molecular weight polyol is said sorbital.

13. The compositon as of claim 1, wherein said aqueous solution is a saline solution with a concentration not larger than 10 wt %.

14. The composition as of claim 1, wherein said composition is a pharmaceutical composition.

15. The composition as of claim 1, wherein said composition is a haircare composition.

16. The composition as of claim 1, wherein said composition is a cosmetic or skincare composition.
